(19) United States
(12) Patent Application Publication
(10) Pub. No.: US 2020/0078481 A1
(43) Pub. Date: Mar. 12, 2020

(54) Display Height-increasing Platform with Keyboard Sterilization Function

(71) Applicant: Loctek Inc.

(72) Inventors: LEHONG XIANG, NINGBO (CN); HAOYU YE, NINGBO (CN)

(73) Assignee: Loctek Inc.

(21) Appl. No.: 16/519,499

(22) Filed: Jul. 23, 2019

(30) Foreign Application Priority Data
Sep. 12, 2018 (CN) 201811066623.3

(51) Int. Cl. A61L 2/10; F16M 11/22; F16M 11/24

(52) U.S. Cl. CPC A61L 2/10; F16M 11/24; F16M 11/22

(57) ABSTRACT

A display height-increasing platform with keyboard sterilization function, comprising a height-increasing platform body, a tablet top arranged on the upper end surface of the height-increasing platform body for supporting the display, a space formed in the lower portion of the height-increasing platform body for the placement of the keyboard; the space is provided with an ultraviolet disinfection unit, which is used for projecting the ultraviolet light onto the upper surface of the keyboard for sterilizing and disinfecting. With an internal ultraviolet disinfection unit in the platform, the keyboard can be disinfected and its cleanliness ensured as the breeding of bacteria avoided.

### CROSS-REFERENCE TO RELATED APPLICATIONS

This application claims priority to Chinese Patent Application No. 201811066623.3 with a filing date of Sep. 12, 2018. The content of the aforementioned application, including any intervening amendments thereto, is incorporated herein by reference.

### TECHNICAL FIELD

The present disclosure relates to the field of supporting devices for computer display, and more particularly, to a display height-increasing platform with keyboard sterilization function.

### BACKGROUND

More and more cervical discomforts occur due to improper postures such as long-term looking down to the computer display, as people are inseparable from the computer in modern life and work; in order to enable the display to rise to a comfortable height for users, there are height-increasing platforms on the market to achieve that, however, these height-increasing platforms are usually designed solely for the lifting purpose, thus single functioned.

### SUMMARY

One objective of the present disclosure is to solve the problem of the prior arts by providing a display height-increasing platform with keyboard sterilization function. With an internal ultraviolet disinfection unit in the platform, the keyboard can be disinfected and its cleanliness ensured as the breeding of bacteria avoided.

The purpose of the present disclosure is realized by:

Providing a display height-increasing platform with keyboard sterilization function, comprising a height-increasing platform body, a tablet top arranged on the upper end surface of the height-increasing platform body for supporting the display, a space formed in the lower portion of the height-increasing platform body for the placement of the keyboard; the space is provided with an ultraviolet disinfection unit, which is used for projecting the ultraviolet light onto the upper surface of the keyboard for sterilizing and disinfecting.

In one embodiment of the display height-increasing platform with keyboard sterilization function of the present disclosure, a display module is installed on the front side of the display height-increasing platform body for indicating the sterilization data information, and a main control module is arranged in the height-increasing platform body, the ultraviolet disinfection unit and the display module are electrically connected with the main control module.

In one embodiment of the display height-increasing platform with keyboard sterilization function of the present disclosure, the display module is provided with a virtual control button, which is used as a control input interface for controlling the ultraviolet disinfection unit.

In one embodiment of the display height-increasing platform with keyboard sterilization function of the present disclosure, a timing module is further provided in the height-increasing platform body, the timing module is electrically connected with the main control module, and is used for turning off the ultraviolet disinfection unit after the ultraviolet disinfection unit has been working for a period of time.

In one embodiment of the display height-increasing platform with keyboard sterilization function of the present disclosure, the space is provided with a supporting plate which is slidably connected inside the space.

In one embodiment of the display height-increasing platform with keyboard sterilization function of the present disclosure, a baffle plate is arranged on one side of the supporting plate, the side of which close to the user; the baffle plate is used for shielding ultraviolet light in the space after the supporting plate slides into the space so as to prevent ultraviolet light from leaking out.

In one embodiment of the display height-increasing platform with keyboard sterilization function of the present disclosure, the baffle plate is movably connected with the supporting plate.

In one embodiment of the display height-increasing platform with keyboard sterilization function of the present disclosure, the baffle plate is rotationally connected with the supporting plate.

In one embodiment of the display height-increasing platform with keyboard sterilization function of the present disclosure, a sliding rail is mounted respectively on both sides of the outer wall of the supporting plate, and the sliding rails on both sides of the supporting plate are respectively fixed with one inner side wall of the space.

In one embodiment of the display height-increasing platform with keyboard sterilization function of the present disclosure, a control module used for controlling the ultraviolet disinfection unit is further arranged in the display height-increasing platform body, and the ultraviolet disinfection unit is an ultraviolet LED lamp.

In one embodiment of the display height-increasing platform with keyboard sterilization function of the present disclosure, a bottom foot is arranged below the display height-increasing platform body, and a space for containing the keyboard is formed between the bottom foot and the body.

The ultraviolet disinfection unit of the present disclosure within the platform is able to disinfect the keyboard so that the keyboard is prevented from the breeding of the bacteria, thus the cleanliness of the keyboard can be ensured.

### BRIEF DESCRIPTION OF THE DRAWINGS

The skilled in the art would more profoundly understand the technical solution of the present disclosure upon reading the following embodiment with reference taken to the accompanying drawings, in which:

FIG. 1 is a schematic perspective diagram of the first embodiment of the display height-increasing platform;

FIG. 2 is a schematic perspective diagram of the first embodiment of the display height-increasing platform;

FIG. 3 is a schematic perspective diagram of the first embodiment of the display height-increasing platform;

FIG. 4 is a schematic perspective diagram of the second embodiment of the display height-increasing platform;

FIG. 5 is a schematic perspective diagram of the third embodiment of the display height-increasing platform.

### EMBODIMENTS

Various aspects of the illustrative embodiments of the present disclosure will be described herein using terms commonly employed by those skilled in the art. Alternate embodiments may be practiced by the skilled in the art under the scope and the principle of the disclosed embodiments. In other instances, well-known features are omitted or simplified in order not to obscure the illustrative embodiments.

Although certain embodiments have been illustrated and described herein for purposes of description, a wide variety of alternate and/or equivalent embodiments or implementations to achieve the same purposes may be substituted for the embodiments shown and described without departing from the scope of the present disclosure. This application is intended to cover any adaptations or variations of the embodiments discussed herein.

### Embodiment 1

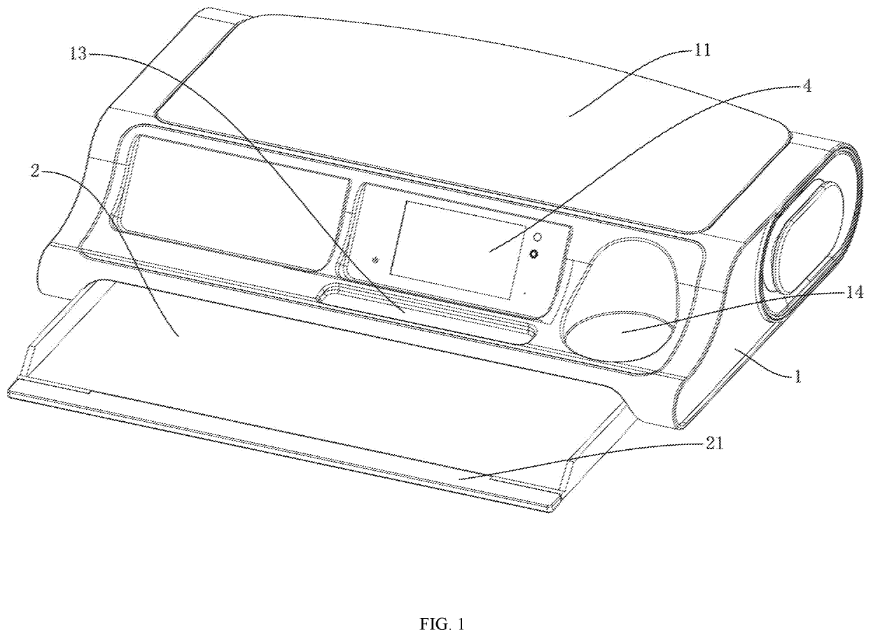

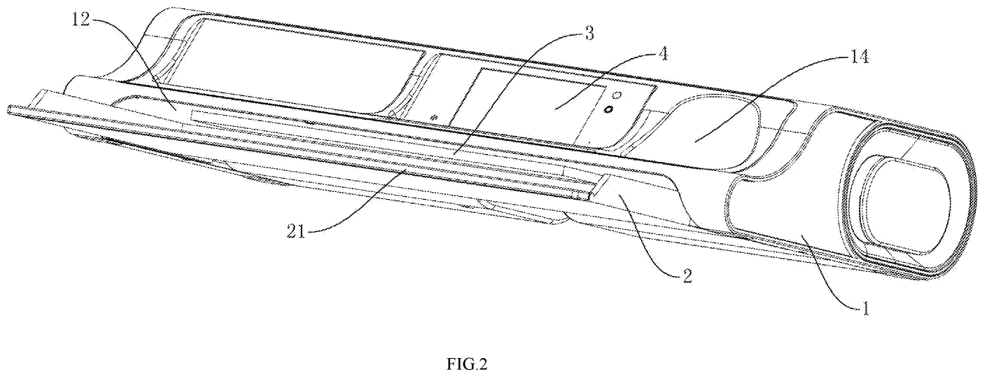

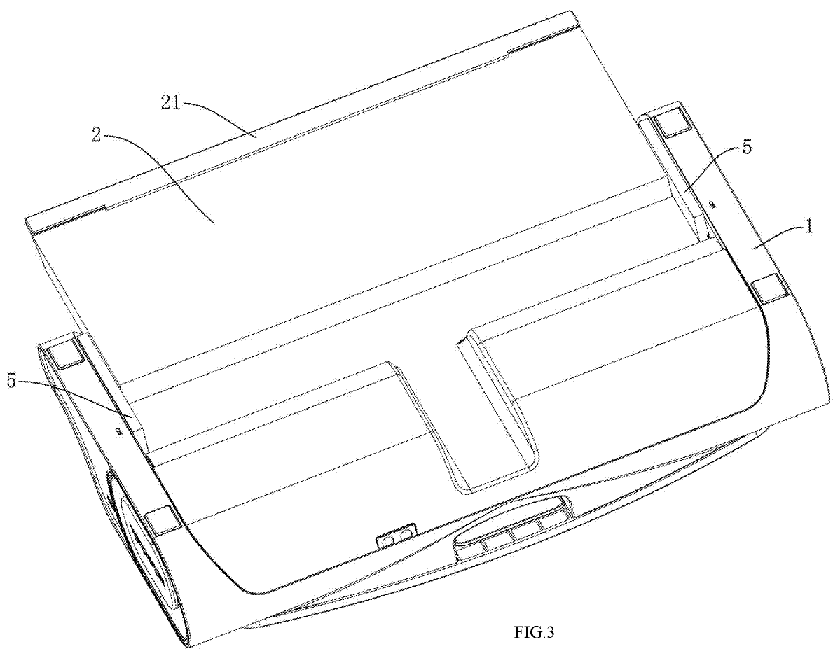

As shown in FIG. 1-3, a display height-increasing platform with keyboard sterilization function, comprising a height-increasing platform body 1, a tablet top 11 arranged on the upper end surface of the height-increasing platform body 1 for supporting the display, a space 12 formed in the lower portion of the height-increasing platform body 1 for the placement of the keyboard; inside the space 12 an ultraviolet disinfection unit 3 is provided, the space 12 may be, for example, of a structure of an accommodating groove, or a grooving structure at the bottom of the height-increasing platform body 1; the ultraviolet disinfection unit 3 is used for projecting the ultraviolet light onto the upper surface of the keyboard for sterilizing and disinfecting; a display module 4 is installed on the front side of the display height-increasing platform body 1 for indicating the sterilization data information, and a main control module is arranged inside the height-increasing platform body 1, the ultraviolet disinfection unit 3 and the display module 4 are electrically connected with the main control module; the sterilization data information displayed by the display module comprises the time used for sterilization, the time remaining for sterilization and etc.; the display module 4 is provided with a virtual control button, which is used as a control input interface for controlling the ultraviolet disinfection unit 3; and the display module is of a touch screen type, so that the sterilization time can be set and the turn-on and turn-off of the ultraviolet disinfection unit 3 can be controlled through the display module 4; a timing module is further provided in the height-increasing platform body 1; the timing module is electrically connected with the main control module, and is used for turning off the ultraviolet disinfection unit 3 after the ultraviolet disinfection unit has been working for a period of time; the timing module enables an auto turning off of the ultraviolet disinfection unit 3 after a certain time of usage, this certain time period such as half an hour, one hour and the like can be set by the user through the display module 4; the space 12 is provided with a supporting plate 2 which is slidably connected inside the space 12; a baffle plate 21 is arranged on one side of the supporting plate 2, the side of which close to the user; the baffle plate 21 is used for shielding ultraviolet light in the space 12 after the supporting plate 2 slides into the space 12 so as to prevent the ultraviolet light from leaking out; the baffle plate 21 is movably connected with the supporting plate 2; the baffle plate 21 is rotationally connected with the supporting plate 2; after the baffle plate 21 is turned upwards, the ultraviolet light in the space can be shielded from escaping out of the space 12; a sliding rail 5 is mounted respectively on both sides of the outer wall of the supporting plate 2 and the sliding rails 5 on both sides of the supporting plate 2 are respectively fixed with one inner side wall of the space 12; with this structure, the supporting plate 2 can be reliably connected in the space 12 in a sliding mode, and a pen groove 13 is formed in the front side of the height-increasing platform body 1, thus it is convenient for the user to place the pen in the pen groove; a cup groove 14 is also formed in the front side of the height-increasing platform body 1, thus it is convenient for the user to place the cup in the cup groove; a control module used for controlling the ultraviolet disinfection unit 3 is further arranged in the display height-increasing platform body 1; the control module is used for controlling the power, opening time of the ultraviolet disinfection unit 3 and the like; and the ultraviolet disinfection unit 3 is an ultraviolet LED lamp; a bottom foot is arranged below the display height-increasing platform body 1, and a space 12 for containing the keyboard is formed between the bottom foot and the body; the ultraviolet disinfection unit 3 of the present disclosure can be used for disinfecting a keyboard placed on the supporting plate 2, so that the keyboard can be prevented from bacteria breeding, and the cleanliness of the keyboard can be ensured.

### Embodiment 2

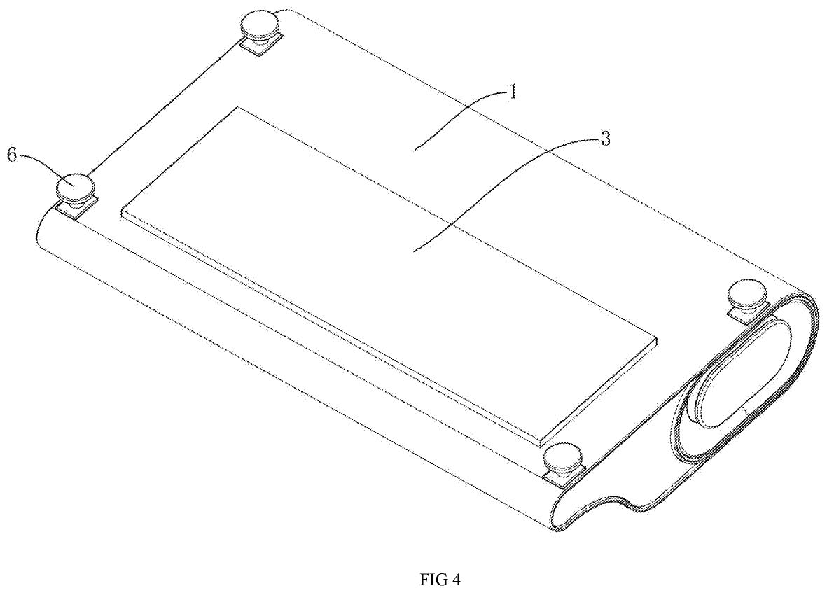

As shown in FIG. 4, different from the first embodiment, the ultraviolet disinfection unit 3 is directly installed on the bottom surface of the display height-increasing platform body 1, and a gap can be generated between the bottom surface of the display height-increasing platform body 1 and the desktop after the installation of supporting foot 6 at the bottom of the display height-increasing platform body 1, and a space for inserting the keyboard is formed between the display height-increasing platform body 1 and the desktop; there is sufficient distance reserved between the ultraviolet disinfection unit 3 and the keyboard to prevent the keyboard from interfering with the ultraviolet disinfection unit 3 after the keyboard inserting into the space; the ultraviolet disinfection unit 3 realizes the disinfection and sterilization of the keyboard after the keyboard inserting into the space; the ultraviolet disinfection unit 3 is an ultraviolet LED lamp in the present disclosure, the ultraviolet LED lamp realizes the disinfection and sterilization of the keyboard; with this structure, a supporting plate structure used for placing a keyboard can be saved in the present display height-increasing platform.

### Embodiment 3

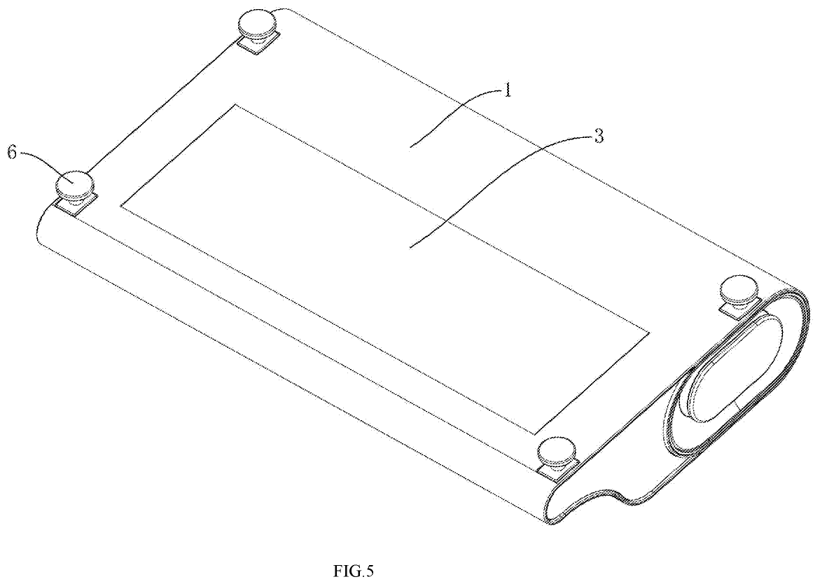

As shown in FIG. 5, different from the first embodiment, the ultraviolet disinfection unit 3 is directly embedded at the bottom surface of the display height-increasing platform body 1; the lower surface of the ultraviolet disinfection unit 3 is at the same height with the lower surface of the display height-increasing platform body 1, a space for inserting the keyboard is formed between the display height-increasing platform body 1 and the desktop after the installation of supporting foot 6 at the bottom of the display height-increasing platform body 1; the ultraviolet disinfection unit 3 realizes the disinfection and sterilization of the keyboard after the keyboard inserting into the space; the ultraviolet disinfection unit 3 is an ultraviolet LED lamp in the present disclosure, the ultraviolet LED lamp realizes the disinfection and sterilization of the keyboard; with this structure, a supporting plate structure used for placing a keyboard can be saved in the present display height-increasing platform.

It should be noted that in the above embodiments, permutation and combination can be performed as long as the technical scheme is not contradicted, those skill in the art can use for exhaustion all possible variations according to the mathematical knowledge of permutation and combination according to the technical scheme, therefore the technical schemes with more arrangement and combination are not described herein. However, it should be understood that the technical schemes after permutation and combination should be deemed as disclosed by the present disclosure.

In conclusion, all the modifications that can directly derived or associated from the content disclosed in the present disclosure by those skill in the art do not depart from the spirit and scope of the technical solutions of the embodiments of the present disclosure.

We claim:

1. A display height-increasing platform with keyboard sterilization function, comprising a height-increasing platform body (1), a tablet top (11) arranged on the upper end surface of the height-increasing platform body (1) for supporting the display, a space (12) formed in the lower portion of the height-increasing platform body (1) for the placement of the keyboard; the space (12) is provided with an ultraviolet disinfection unit (3); the ultraviolet disinfection unit (3) is used for projecting the ultraviolet light onto the upper surface of the keyboard for sterilizing and disinfecting.

2. The display height-increasing platform with keyboard sterilization function of claim 1, wherein a display module (4) is installed on the front side of the display height-increasing platform body (1) for indicating the sterilization data information, and a main control module is arranged in the height-increasing platform body (1), the ultraviolet disinfection unit (3) and the display module (4) are electrically connected with the main control module.

3. The display height-increasing platform with keyboard sterilization function of claim 2, wherein the display module (4) is provided with a virtual control button, which is used as a control input interface for controlling the ultraviolet disinfection unit (3).

4. The display height-increasing platform with keyboard sterilization function of claim 2, wherein a timing module is further provided in the height-increasing platform body (1), the timing module is electrically connected with the main control module, and is used for turning off the ultraviolet disinfection unit (3) after the ultraviolet disinfection unit has been working for a period of time.

5. The display height-increasing platform with keyboard sterilization function of claim 1, wherein the space (12) is provided with a supporting plate (2) which is slidably connected inside the space (12).

6. The display height-increasing platform with keyboard sterilization function of claim 5, wherein a baffle plate (21) is arranged on one side of the supporting plate (2), the side of which close to the user; the baffle plate (21) is used for shielding ultraviolet light in the space (12) after the supporting plate (2) slides into the space (12) so as to prevent ultraviolet light from leaking out.

7. The display height-increasing platform with keyboard sterilization function of claim 6, wherein the baffle plate (21) is movably connected with the supporting plate (2).

8. The display height-increasing platform with keyboard sterilization function of claim 5, wherein a sliding rail (5) is mounted respectively on both sides of the outer wall of the supporting plate (2), and the sliding rails (5) on both sides of the supporting plate (2) are respectively fixed with one inner side wall of the space (12).

9. The display height-increasing platform with keyboard sterilization function of claim 1, wherein a control module used for controlling the ultraviolet disinfection unit (3) is further arranged in the display height-increasing platform body (1), and the ultraviolet disinfection unit (3) is an ultraviolet LED lamp.

10. The display height-increasing platform with keyboard sterilization function of claim 1, wherein a bottom foot is arranged below the display height-increasing platform body (1), and a space (12) for containing the keyboard is formed between the bottom foot and the body.

11. The display height-increasing platform with keyboard sterilization function of claim 2, wherein the space (12) is provided with a supporting plate (2) which is slidably connected inside the space (12).

12. The display height-increasing platform with keyboard sterilization function of claim 11, wherein a baffle plate (21) is arranged on one side of the supporting plate (2), the side of which close to the user; the baffle plate (21) is used for shielding ultraviolet light in the space (12) after the supporting plate (2) slides into the space (12) so as to prevent ultraviolet light from leaking out.

13. The display height-increasing platform with keyboard sterilization function of claim 12, wherein the baffle plate (21) is movably connected with the supporting plate (2).

14. The display height-increasing platform with keyboard sterilization function of claim 11, wherein a sliding rail (5) is mounted respectively on both sides of the outer wall of the supporting plate (2), and the sliding rails (5) on both sides of the supporting plate (2) are respectively fixed with one inner side wall of the space (12).

15. The display height-increasing platform with keyboard sterilization function of claim 3, wherein the space (12) is provided with a supporting plate (2) which is slidably connected inside the space (12).

16. The display height-increasing platform with keyboard sterilization function of claim 15, wherein a baffle plate (21) is arranged on one side of the supporting plate (2), the side of which close to the user; the baffle plate (21) is used for shielding ultraviolet light in the space (12) after the supporting plate (2) slides into the space (12) so as to prevent ultraviolet light from leaking out.

17. The display height-increasing platform with keyboard sterilization function of claim 16, wherein the baffle plate (21) is movably connected with the supporting plate (2).

18. The display height-increasing platform with keyboard sterilization function of claim 15, wherein a sliding rail (5) is mounted respectively on both sides of the outer wall of the supporting plate (2), and the sliding rails (5) on both sides of the supporting plate (2) are respectively fixed with one inner side wall of the space (12).
